(19) United States
(12) Patent Application Publication
(10) Pub. No.: US 2026/0215712 A1
(43) Pub. Date: Jul. 30, 2026

(54) INFORMATION PROCESSING DEVICE, INFORMATION PROCESSING METHOD, AND RECORDING MEDIUM

(71) Applicant: NEC Corporation, Tokyo (JP)

(72) Inventors: Yoshikazu ARAI, Tokyo (JP); Tsuyoshi NAKAMURA, Tokyo (JP)

(73) Assignee: NEC Corporation, Tokyo (JP)

(21) Appl. No.: 19/450,981

(22) Filed: Jan. 16, 2026

(30) Foreign Application Priority Data
Jan. 30, 2025 (JP) 2025-013523

(51) Int. Cl. A61B 5/16; A61B 5/00; A61B 5/11; G06T 7/00; G06T 7/246; G10L 17/02; G10L 25/66

(52) U.S. Cl. CPC A61B 5/162; A61B 5/1103; A61B 5/163; A61B 5/4809; A61B 5/742; G06T 7/0012; G06T 7/246; G10L 17/02; G10L 25/66; A61B 2503/08; G06T 2207/10016; G06T 2207/30201

(57) ABSTRACT

In order to improve evaluation accuracy of a cognitive function, in an information processing device, a processor transmits dialogue information indicating a dialogue content to be uttered to a subject and acquires reaction information indicating a reaction of the subject to the dialogue content. The processor cuts out pieces of partial information in a plurality of discontinuous portions from the reaction information acquired and generates test data by combining in time series the pieces of partial information cut out. The processor analyzes a cognitive function level of the subject and outputs an analysis result by using the test data generated.

### INCORPORATION BY REFERENCE

This application is based upon and claims the benefit of priority from Japanese Patent Application 2025-013523, filed on Jan. 30, 2025, the disclosure of which is incorporated herein in its entirety by reference.

### TECHNICAL FIELD

The present disclosure relates to a technique for maintaining health of a subject.

### BACKGROUND ART

In recent years, in Japan, the aging rate with respect to the total population increases, and the number of elderly people living alone tends to increase year by year. It is considered that an elderly person living alone has few opportunities to have conversations with family members and the like, and also has a high risk of onset and progression of dementia and a state requiring care due to a decrease in social activity. In particular, various studies have revealed that a decrease in daily conversation amount affects the onset of dementia.

For instance, Patent Document 1 proposes evaluating a cognitive function from a magnitude of a change and/or a temporal change in feature represented by time-series data of one kind of variable reflecting prosodic information in a predetermined extraction period of voice data of a subject.

Patent Document 1: Japanese Patent Application Laid-Open under No. 2017-148431

### SUMMARY

Evaluation accuracy of a cognitive function depends on an acquisition frequency of information such as a voice of a subject in daily life, an amount of acquired information, and the like. The higher the acquisition frequency and the larger the amount of the acquired information, the higher the evaluation accuracy of the cognitive function. However, under the present circumstances, a subject who is concerned about a decline in cognitive function spontaneously evaluates the cognitive function. It is known that a level of the cognitive function fluctuates due to an influence of a mental and physical state of the subject at a time of a test, and in JP 2017-148431 A, there is a case where the cognitive function cannot be evaluated with sufficient accuracy with an acquisition frequency of information regarding the subject and an amount of obtained information.

One of objects of the present disclosure is to improve evaluation accuracy of a cognitive function.

According to an example aspect of the present invention, there is provided an information processing device including:
at least one first memory configured to store first instructions; and
at least one first processor configured to execute the first instructions to:
transmit dialogue information indicating a dialogue content to be uttered to a subject and acquire reaction information indicating a reaction of the subject to the dialogue content;
cut out pieces of partial information in a plurality of discontinuous portions from the reaction information acquired and generate test data by combining in time series the pieces of partial information cut out; and
analyze a cognitive function level of the subject and output an analysis result by using the test data generated.

According to another example aspect of the present invention, there is provided an information processing method performed by a computer, the method including:
transmitting dialogue information indicating a dialogue content to be uttered to a subject and acquiring reaction information indicating a reaction of the subject to the dialogue content;
cutting out pieces of partial information in a plurality of discontinuous portions from the reaction information acquired and generating test data by combining in time series the pieces of partial information cut out; and
analyzing a cognitive function level of the subject and output an analysis result by using the test data generated.

According to a further example aspect of the present invention, there is provided a non-transitory computer-readable recording medium storing a program causing a computer to execute processing of:
transmitting dialogue information indicating a dialogue content to be uttered to a subject and acquiring reaction information indicating a reaction of the subject to the dialogue content;
cutting out pieces of partial information in a plurality of discontinuous portions from the reaction information acquired and generating test data by combining in time series the pieces of partial information cut out; and
analyzing a cognitive function level of the subject and output an analysis result by using the test data generated.

### EFFECT

According to the present disclosure, it is an object to improve evaluation accuracy of a cognitive function.

### BRIEF DESCRIPTION OF THE DRAWINGS

FIG. 1 is an example of a schematic configuration of a cognitive function evaluation system to which an information processing device according to the present disclosure is applied;

FIG. 2 is a block diagram illustrating an example of a hardware configuration of a management server according to the present disclosure;

FIG. 3 is a block diagram illustrating an example of a hardware configuration of a dialogue device according to the present disclosure;

FIG. 4 is a diagram illustrating an example of a functional configuration of the management server according to the present disclosure;

FIG. 5 is a diagram illustrating an example of a functional configuration of the dialogue device according to the present disclosure;

FIG. 6 is a flowchart for illustrating processing in the cognitive function evaluation system according to the present disclosure;

FIG. 7 is a diagram illustrating a generation example of test data;

FIGS. 8A and 8B are graphs illustrating a comparative example of an analysis result of a cognitive function;

FIG. 9 is a block diagram illustrating a functional configuration of another management server according to the present disclosure;

FIG. 10 is a flowchart of processing executed by the another management server according to the present disclosure;

FIG. 11 is a block diagram illustrating a functional configuration of another dialogue device according to the present disclosure; and

FIG. 12 is a flowchart of processing executed by the another dialogue device according to the present disclosure.

### EXAMPLE EMBODIMENTS

Hereinafter, example embodiments of the present disclosure will be described with reference to the drawings. In the present disclosure, a cognitive function of an elderly person is accurately analyzed by eliminating previous preparation of the elderly person by continuously checking the cognitive function in daily life and improving a test frequency in a natural state. A large language model (LLM) technology is utilized to enable an elderly person to continuously check a cognitive function without being conscious, and data representing a natural reaction of the elderly person in daily life is collected to provide an analysis result of the cognitive function of the elderly person.

### First Example Embodiment

### (System Configuration)

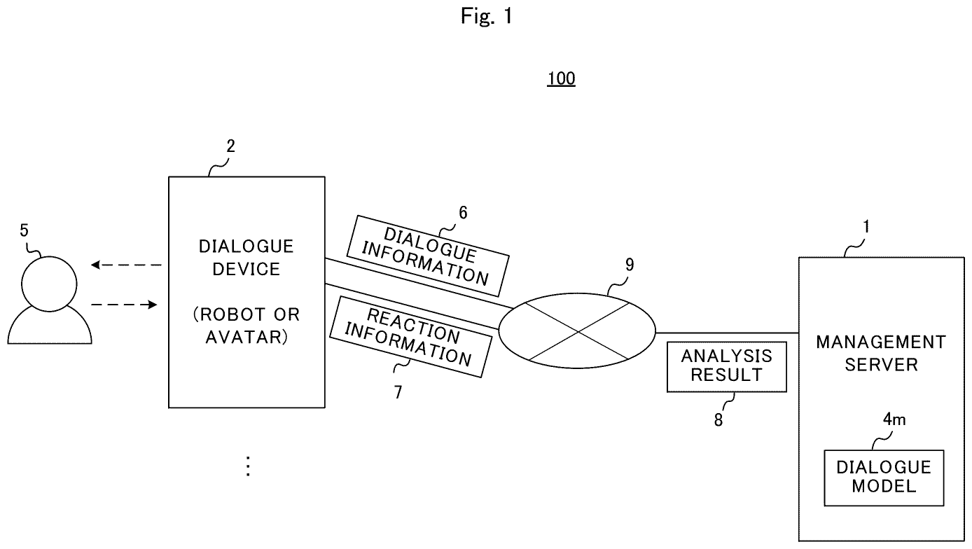

FIG. 1 is an example of a schematic configuration of a cognitive function evaluation system 100 to which an information processing device of the present disclosure is applied. The cognitive function evaluation system 100 is a system that analyzes a cognitive function of an elderly person (Hereinafter, it is referred to as a “subject 5”.) based on information (Hereinafter, it is referred to as “reaction information 7”.) obtained through a dialogue with the subject 5 in daily life. The cognitive function evaluation system 100 includes a management server 1 and one or more dialogue devices 2, and the management server 1 and the dialogue device 2 are communicably connected via a network 9 such as the Internet.

The management server 1 is an information processing device that executes processing, storage, and transmission/reception of various pieces of data. In addition, the management server 1 includes a dialogue model 4m that generates a dialogue timing and a dialogue topic, and conducts a dialogue with the subject 5 via the dialogue device 2. The management server 1 analyzes the cognitive function of the subject 5 by using the reaction information 7 obtained from the dialogue device 2 by the dialogue, and provides an analysis result 8.

The dialogue device 2 is a terminal device such as a smartphone, a tablet, a PC, or a robot used by the subject 5 who is concerned about a decline in the cognitive function, and transmits, to the management server 1, the reaction information 7 obtained by detecting a reaction of the subject 5 to the dialogue generated by the dialogue model 4m. In a case where the dialogue device 2 is the smartphone, the tablet, the PC, or the like, an avatar may be displayed to have a conversation with the subject 5.

Note that the method of providing the reaction information 7 indicating the reaction of the subject 5 to the management server 1 is not limited to the above-described method. For instance, the reaction information 7 may be acquired using an external storage such as a universal serial bus (USB) memory without passing through the network 9 and stored in the management server 1 as appropriate.

### (Hardware Configuration)

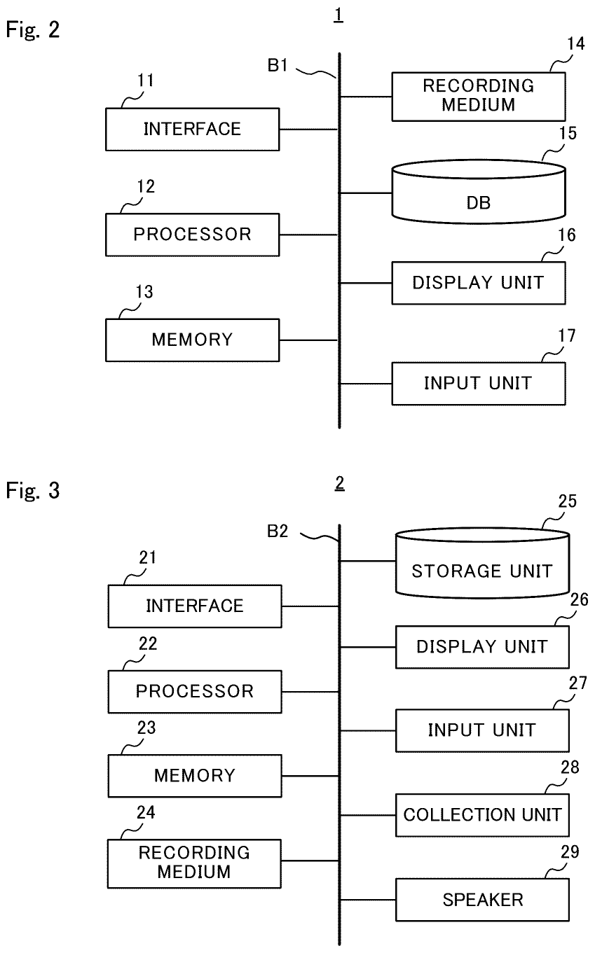

FIG. 2 is a block diagram illustrating an example of a hardware configuration of the management server. As illustrated in FIG. 2, the management server 1 includes an interface 11, a processor 12, a memory 13, a recording medium 14, a database (DB) 15, a display unit 16, and an input unit 17. The interface 11, the processor 12, the memory 13, the recording medium 14, the database (DB) 15, the display unit 16, and the input unit 17 are connected to a bus B2 and can communicate with each other.

The interface 11 exchanges data with the dialogue device 2 and the like. The interface 11 is used to receive the reaction information 7 of the subject 5 from the dialogue device 2 via the network 9, and to transmit and receive data to and from the dialogue device 2 and the like. The interface 11 is also used in a case where the management server 1 exchanges data with a predetermined device connected via the network 9. The predetermined device is a device other than the dialogue device 2, and corresponds to, for instance, a device used by a person who is permitted to view the analysis result 8 of the subject 5 by a predetermined method, such as a family member of the subject 5 and a related person such as a medical worker.

The processor 12 is a computer such as a central processing unit (CPU), and controls the entire management server 1 by executing a program prepared in advance. As the processor 12, a CPU, a graphics processing unit (GPU), a digital signal processor (DSP), a micro processing unit (MPU), a floating point number processing unit (FPU), a physics processing unit (PPU), a tensor processing unit (TPU), a quantum processor, a microcontroller, a combination of these, or the like can be used.

The memory 13 includes a read only memory (ROM), a random access memory (RAM), and the like. The memory 13 stores a program executed by the processor 12. The memory 13 is also used as a work memory during execution of various types of processing by the processor 12.

The recording medium 14 is a non-volatile and non-transitory recording medium such as a disk-shaped recording medium or a semiconductor memory, and is attachable to and detachable from the management server 1. The recording medium 14 records various programs to be executed by the processor 12. In a case where the management server 1 executes processing of analyzing the cognitive function, the program recorded in the recording medium 14 is loaded into the memory 13 and executed by the processor 12.

The DB 15 stores information related to a conversation for each subject 5. The display unit 16 displays a predetermined image by, for instance, a liquid crystal display (LCD). The input unit 17 is a keyboard, a mouse, a touch panel, or the like, and is used by an operator who manages the management server 1.

FIG. 3 is a block diagram illustrating an example of a hardware configuration of the dialogue device. As illustrated in FIG. 3, the dialogue device 2 includes an interface 21, a processor 22, a memory 23, a recording medium 24, a storage unit 25, a display unit 26, an input unit 27, a collection unit 28, and a speaker 29. The interface 21, the processor 22, the memory 23, the recording medium 24, the storage unit 25, the display unit 26, the input unit 27, the collection unit 28, and the speaker 29 are connected to a bus B2 and can communicate with each other.

The interface 21 exchanges data with the management server 1 via the network 9. The interface 21 is used in a case where the reaction information 7 of the subject 5 is transmitted to the management server 1 or the analysis result 8 of the cognitive function of the subject 5 is received from the management server 1.

The processor 22 is a computer such as a CPU, and controls the entire dialogue device 2 by executing a program prepared in advance. As the processor 22, it is possible to use a CPU, a GPU, a DSP, an MPU, an FPU, a PPU, a TPU, a quantum processor, a microcontroller, a combination of these, or the like.

The memory 23 includes a ROM, a RAM, or the like. The memory 23 stores a program executed by the processor 22. The memory 23 is also used as a working memory during execution of various types of processing by the processor 22.

The recording medium 24 is a non-volatile non-transitory recording medium such as a disk-shaped recording medium or a semiconductor memory, and is attachable to and detachable from the dialogue device 2. The recording medium 24 records various programs to be executed by the processor 22. The storage unit 25 accumulates the reaction information 7 indicating the reaction of the subject 5 in daily life. The display unit 26 displays a predetermined image by, for instance, an LCD. The input unit 27 is a touch panel or the like, and is used in a case where the subject 5 performs a predetermined operation. The collection unit 28 includes a device for collecting the reaction information 7 of the subject 5. The speaker 29 is used to output a designated dialogue content by voice or the like in response to an instruction from the processor 22.

Hereinafter, as an example, it is assumed that the cognitive function of the subject 5 is evaluated based on the voice of the subject 5 during conversation. In this example, it is assumed that the collection unit 28 includes a microphone for collecting the voice uttered by the subject 5.

### (Functional Configuration)

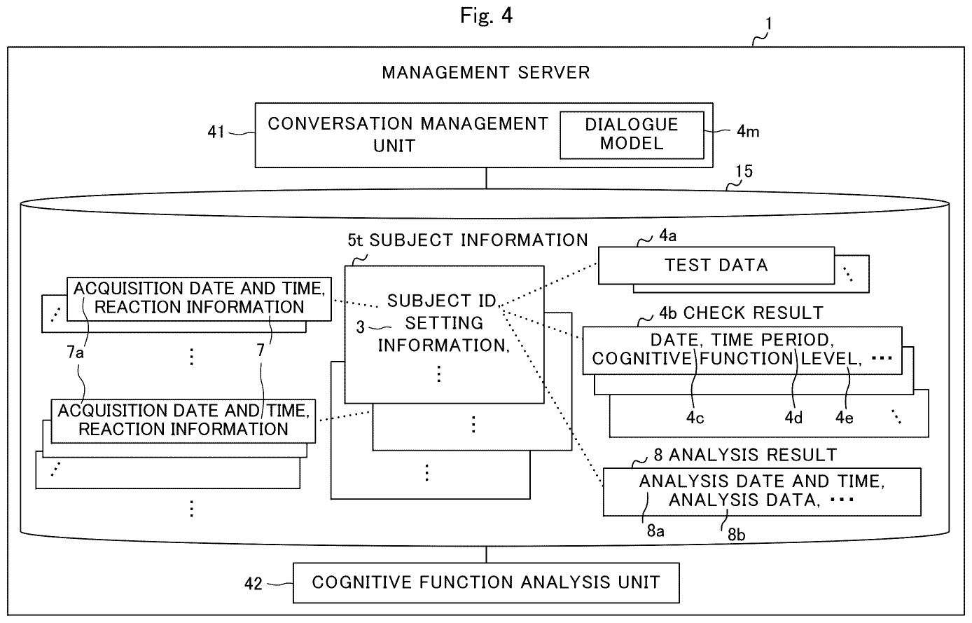

FIG. 4 is a block diagram illustrating an example of a functional configuration of the management server. The management server 1 functionally includes a conversation management unit 41 and a cognitive function analysis unit 42. The conversation management unit 41 and the cognitive function analysis unit 42 are achieved by the processor 12 executing programs each associated to the above-described units.

The conversation management unit 41 transmits, to the dialogue device 2, various topics that are generated by the dialogue model 4m and in which the subject 5 is interested in such a way that the subject 5 can continue conversation on a daily basis. As a result, the dialogue model 4m conducts a dialogue with the subject 5 via the dialogue device 2. In a case where the reaction information 7 is received from the dialogue device 2, the conversation management unit 41 accumulates the reaction information 7 in the DB 15 in association with a subject ID for identifying the subject 5.

The DB 15 is a database that saves various pieces of data for analyzing a cognitive function of each of one or a plurality of subjects 5. The DB 15 stores subject information 5t, the reaction information 7, test data 4a, a check result 4b, the analysis result 8, and the like. The subject information 5t includes the subject ID for identifying the subject 5, setting information 3 related to a dialogue, and the like. The reaction information 7, the test data 4a, the check result 4b, and the analysis result 8 are associated with the subject ID of the subject information 5t. The setting information 3 includes a time period in which a dialogue is conducted, a predetermined amount required for analyzing the cognitive function, and the like.

The reaction information 7 corresponds to voice information representing a voice of the subject 5 recorded during conversation, and in a case where the reaction information 7 is transmitted to the management server 1, an acquisition date and time 7a in a case where the dialogue device 2 has acquired the conversation voice of the subject 5 is added. The test data 4a, the check result 4b, and the analysis result 8 are information generated and stored by processing of analyzing the cognitive function (Hereinafter, it is referred to as “cognitive function analysis processing”.).

The cognitive function analysis unit 42 executes the cognitive function analysis processing for each subject 5. The cognitive function analysis processing includes cognitive function check processing based on the accumulated reaction information 7 and tendency analysis processing of analyzing a tendency of the cognitive function based on a plurality of check results 4b obtained up to the present by the cognitive function check processing.

The cognitive function check processing is relevant to a function capable of testing mild cognitive impairment (MCI). As the cognitive function check processing, for instance, a technology of analyzing a voiceprint waveform characteristically seen in MCI from the acquired voice information is used. As an example, it is conceivable to use the technology of Canary Speech, Inc. In this case, in a case where a total time of voice information obtained from the same subject 5 is 40 seconds or more, the cognitive function can be analyzed. Here, it is sufficient that the total time of the accumulated voice information is 40 second or more, and 40 seconds or more is not required for a piece of voice information. Any technology capable of testing MCI can be used, and is not limited to the technology of Canary Speech, Inc. A voice information amount to be tested may be set to 40 seconds or less from a minimum value of the voice information amount required for the test, a voice information amount according to desired accuracy, and the like according to an applied technology, and is not limited to 40 seconds or more described above.

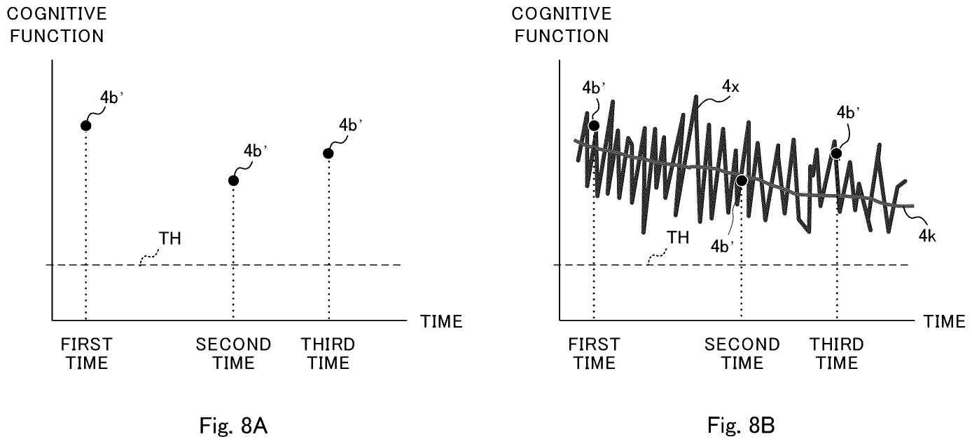

In the tendency analysis processing, as an example, an approximate curve 4k (FIG. 8B) is calculated using a scatter diagram in which values of MCI are plotted. A calculation method performed in the tendency analysis processing is not limited to a method of calculating the approximate curve 4k by using the scatter diagram, as long as the subject 5 or the like can understand a tendency of maintenance, improvement, or decline in the cognitive function of the subject 5. An example of the approximate curve 4k using the scatter diagram will be described later. The analysis result 8 generated by the tendency analysis processing includes an analysis date and time 8a, analysis data 8b, and the like.

Here, the dialogue model 4m learns a dialogue timing at which a conversation with the subject 5 can be continued and the test data 4a of a predetermined amount or more can be generated, in each of a plurality of time periods. In addition, the dialogue model 4m generates a topic that allows conversation to be continued, such as a topic related to expertise of the subject 5, a personal topic, a topic related to a past dialogue content based on the accumulated reaction information 7, or a topic for encouraging feeling expression. The dialogue timing is recorded in the setting information 3. The predetermined amount can be, for instance, 40 seconds or more in a case where the technology of Canary Speech, Inc. is used. In addition, the number of time periods may be different for each day of the week.

The cognitive function analysis unit 42 executes the cognitive function analysis processing of analyzing the reaction information 7 accumulated in the DB 15 at an analysis timing of analyzing the cognitive function, and stores the analysis result 8 obtained by the cognitive function analysis processing in the DB 15. The analysis timing is different from the dialogue timing determined for each time period, and can be every end time of the time period, every day, every week, every month, or the like. Then, the newly obtained analysis result 8 is transmitted to the dialogue device 2 via the network 9. Furthermore, the analysis result 8 may be notified to the subject 5, a person related to the subject 5, or the like by e-mail.

In the above configuration, the conversation management unit 41 of the management server 1 is an example of a reaction information acquisition means of the present disclosure, and the cognitive function analysis unit 42 is an example of a test data generation means and an analysis means of the present disclosure.

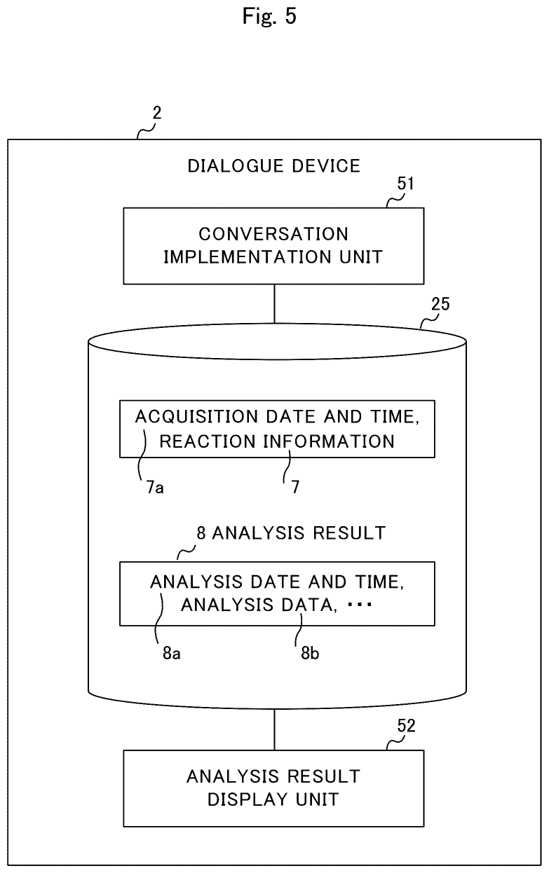

FIG. 5 is a block diagram illustrating an example of a functional configuration of the dialogue device. The dialogue device 2 functionally includes a conversation implementation unit 51 and an analysis result display unit 52. The conversation implementation unit 51 and the analysis result display unit 52 are achieved by the processor 22 executing programs each associated to the above-described units.

The conversation implementation unit 51 has a conversation with the subject 5 via the dialogue device 2 by using dialogue information 6 indicating the dialogue content generated by the dialogue model 4m. As an example, according to the dialogue information 6 generated by the dialogue model 4m in the morning, the daytime, and the evening, the conversation implementation unit 51 asks a question such as “Good morning! What are your plans for today?” in the morning hours, asks a question such as “Are you going to shop today?” in the daytime hours, and asks a question such as “Which would you like, today's news or weather forecast?” in the evening hours, encouraging the subject 5 to have a dialogue with the dialogue device 2. In addition, in a case where there is an answer from the subject 5, another question may be asked to continue the conversation in response to reception of the dialogue information 6 further generated by the dialogue model 4m. The reaction information 7 including voice information obtained in this manner during the conversation and the acquisition date and time 7a of the voice information is temporarily held in the storage unit 25 and sequentially transmitted to the management server 1 by the conversation implementation unit 51.

In a case of receiving the analysis result 8 from the management server 1, the analysis result display unit 52 stores the analysis result 8 in the storage unit 25, and displays the received analysis result 8 on the display unit 26. As an example, the analysis result display unit 52 displays the graph illustrated in FIG. 8B by using the analysis data 8b included in the analysis result 8. Notification of the analysis result 8 to the subject 5 is not limited to the display of the analysis result 8 on the display unit 26. The notification may be made using a mail address of the subject 5 or a related person by a mechanism of the management server 1. Alternatively, the conversation implementation unit 51 may start a conversation regarding the reception of the analysis result 8.

### (Processing Flow)

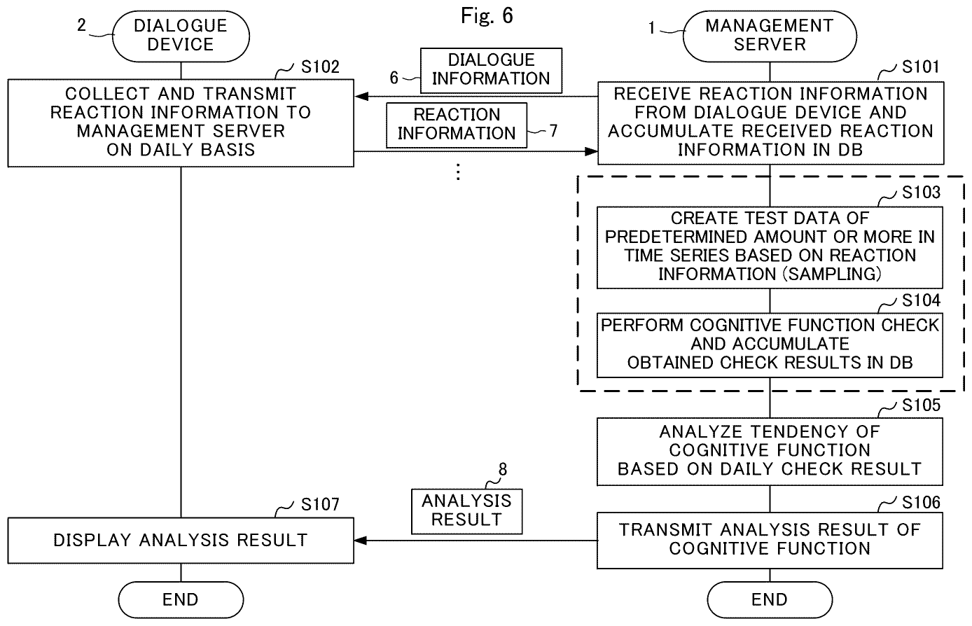

FIG. 6 is a flowchart for illustrating the cognitive function analysis processing in the cognitive function evaluation system. In FIG. 6, in daily life, the management server 1 transmits the dialogue information 6 generated by the dialogue model 4m to the dialogue device 2. In a case where the reaction information 7 is received from the dialogue device 2, the conversation management unit 41 accumulates the received reaction information 7 in the DB 15 in association with the subject ID of the subject 5 of the dialogue device 2 (step S101). In the dialogue device 2, every time the conversation implementation unit 51 receives the dialogue information 6 generated by the dialogue model 4m, a dialogue content based on the dialogue information 6 is output from the speaker 29 to have a conversation with the subject 5, and the reaction information 7 of the subject 5 is acquired by the collection unit 28 and sequentially transmitted to the management server 1 (step S102).

Then, in the management server 1, the cognitive function analysis unit 42 repeats execution of the cognitive function check processing by using the reaction information 7 for each time period indicated by the setting information 3 and processing of accumulating the check results 4b in the DB 15 (steps S103 and S104). Hereinafter, steps S103 and S104 will be described in detail.

First, the cognitive function analysis unit 42 creates the time-series test data 4a of a predetermined amount or more by using the reaction information 7 that has not undergone the cognitive function check processing from the accumulated reaction information 7 (step S103). As an example, the test data 4a is created for each time period designated in the setting information 3. Then, the created test data 4a is stored in the DB 15 in association with the subject ID.

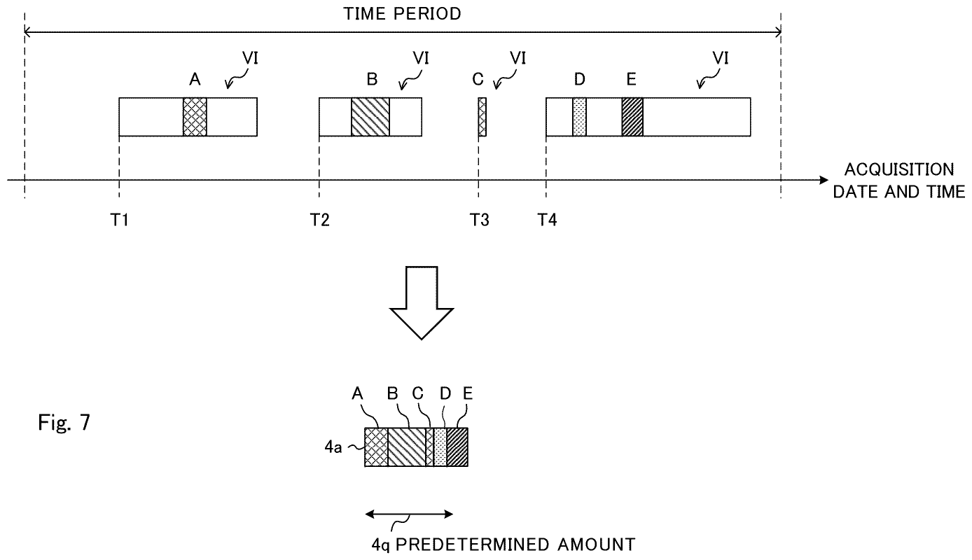

Here, as illustrated in FIG. 7, it is sufficient that the test data 4a is data of a predetermined amount 4q or more and is, for instance, data in which partial information is cut out from a plurality of portions of one or more pieces of voice information received as the reaction information 7 and the cutout portions are combined in time series. FIG. 7 illustrates a case where conversation is performed four times at times T1, T2, T3, and T4 in a certain time period (for instance, daytime hours), and four pieces of voice information VI are acquired at that time. The times T1, T2, T3, and T4 are values indicated by the acquisition date and time 7a. The cognitive function analysis unit 42 cuts out voiceprint portions suitable for the MCI test from each of the pieces of voice information VI as pieces of partial information A, B, C, D, and E, and combines them in time series, thereby generating the test data 4a of the predetermined amount 4q (for instance, 40 seconds) or more. Note that the cutout of the voiceprint portions (Hereinafter, it is referred to as “sampling”.) is performed by similarity determination with a waveform suitable for the MCI test, and does not analyze the content of the conversation uttered by the subject 5.

Subsequently, the cognitive function analysis unit 42 executes the cognitive function check processing by using the created test data 4a, and accumulates the obtained check results 4b in the DB 15 (step s104). For instance, the check result 4b for each time period designated in the setting information 3 is accumulated. In this case, the cognitive function analysis unit 42 accumulates the check results 4b indicating a date 4c, a time period 4d, a cognitive function level 4e, and the like in the DB 15.

In a case where the cognitive function analysis unit 42 has checked all the reaction information 7 in a date and time period to be processed, the reaction information 7 in a next time period is to be checked in time series, and the processing of step S104 is repeated. In a case of obtaining the check results 4b for all the dates and time periods, the cognitive function analysis unit 42 ends the cognitive function check processing, and proceeds to step S105 to analyze the tendency of the cognitive function. That is, the repetitive processing of steps S103 and S104 ends. At this point, for the same date 4c, for instance, three check results 4b of the morning, the daytime, and the evening are created.

Next, the cognitive function analysis unit 42 analyzes the tendency of the daily cognitive function based on the check results 4b accumulated in the DB 15, and evaluates the current cognitive function level 4e of the subject 5 (step S105). For instance, the cognitive function analysis unit 42 analyzes the tendency of the cognitive function by using all of the check results 4b or the check result 4b for a certain period from the latest (for instance, at the start of the cognitive function, 1 year, or 2 years, etc.). As an analysis method, for instance, the approximate curve 4k (FIG. 8B) representing the tendency can be calculated using the scatter diagram in which the cognitive function levels 4e (that is, the values of MCI) are plotted, based on the plurality of check results 4b. The analysis result 8 obtained in this manner is stored in the DB 15. In addition, the cognitive function analysis unit 42 transmits the analysis result 8 to the dialogue device 2 of the subject 5 (step S106), and thus, the analysis result 8 is displayed on the dialogue device 2 (step S107). The cognitive function analysis unit 42 may transmit the analysis result 8 to the subject 5, a related person, or the like by using a mail address designated in advance. The cognitive function analysis unit 42 may calculate the approximate curve 4k for each of the morning, the daytime, and the evening. In this case, it is possible to know the tendency of the cognitive function for each time period.

### (Dialogue Model)

The dialogue model 4m is a model that performs machine learning in order to estimate a dialogue timing and a dialogue content for talking to the subject 5. An average dialogue timing at which a conversation is generally likely to occur is set as an initial timing for each time period, the dialogue timing is changed using a predetermined algorithm, and one or a plurality of times at which the voice information becomes information of a predetermined amount or more is learned for each time period. As an example, as an example of a plurality of time periods, in a case where the morning hours are set to 8:00 to 13:00, the daytime hours are set to 13:00 to 18:00, and the night hours are set to 18:00 to 23:00, the dialogue timing is learned in such a way that the test data 4a becomes data of the predetermined amount (for instance, 40 seconds) or more in each time period. The dialogue model 4m may learn the number of time periods for each day of the week. In this case, the number of time periods is adjusted for each day of the week.

The dialogue timing is determined using the trained dialogue model 4m. The conversation management unit 41 talks to the subject 5 via the dialogue device 2 at the timing determined by the dialogue model 4m. A weight of the dialogue model 4m is adjusted according to a difference between the predetermined amount and the amount of the obtained voice information. By using the dialogue model 4m, the conversation management unit 41 causes the dialogue device 2 to continuously conduct a dialogue, makes it a habit that the subject 5 has a conversation, and also eliminates previous preparation of the subject 5, in such a way that the test in a natural state can be performed. In addition, the conversation with the subject 5 becomes possible until the amount of the voice information of the subject 5 reaches at least the predetermined amount daily in each time period of the morning, the daytime, and the evening. In this case, dialogue is conducted at least three times per day.

FIG. 8A and FIG. 8B are graphs illustrating a comparative example of the analysis result of the cognitive function. In the graphs of FIG. 8A and FIG. 8B, the vertical axis represents a level of the cognitive function, and the horizontal axis represents the lapse of time. It is determined that there is no tendency of MCI as the cognitive function is higher. A value for determining that the cognitive function is at a level suspected of MCI is indicated by a threshold TH. In a case where the value falls below the threshold TH, it indicates that the cognitive function reaches MCI.

FIG. 8A illustrates check results 4b′ in a case where the subject 5 visits a doctor and undergoes a cognitive function test. In this example, the subject 5 visits the doctor three times, but the visit interval is not constant. In addition, the second check result 4b′ is lower than the first check result 4b′, but the third check result 4b′ is increased to substantially midway between the first check result 4b′ and the second check result 4b′. However, the cognitive function fluctuates under an influence of a daily change in physical condition. In addition, a frequency of the visit of the subject 5 is low, and the visit interval is not constant. Therefore, in FIG. 8A, it is difficult to determine whether the cognitive function tends to decline or is affected by the change in physical condition.

FIG. 8B illustrates an example of the check results in a case where the technique of the present disclosure is used. This example shows a fluctuation 4x of the cognitive function level 4e based on the check results 4b′ of the subject 5 by the three visits to the doctor illustrated in FIG. 8A and further the check results 4b obtained using the technique of the present disclosure. The fluctuation 4x visualizes a change in the daily cognitive function level 4e. In addition, in the technique of the present disclosure, the approximate curve 4k is illustrated in which the tendency of the cognitive function level 4e is calculated based on check results 4b′ and the check results 4b. By visually confirming the approximate curve 4k, the decline tendency of the cognitive function can be easily understood with high accuracy.

As described above, according to the present disclosure, the subject 5 is habituated to have a dialogue with the dialogue device 2 on a daily basis, and the subject 5 can continuously check the cognitive function without being conscious. In addition, by habituating daily conversation with the dialogue device 2, it is possible to suppress the decline in the cognitive function of the subject 5 and to check the cognitive function of the subject 5 in a natural state. Furthermore, the frequency of checking the cognitive function can be increased as compared with a case of visiting a doctor.

Furthermore, according to the present disclosure, the cognitive function is checked based on the voiceprint, not based on the content of the conversation uttered by the subject 5. Specifically, it is known that a frequency characteristic (spectrum) of a voice uttered by a person whose cognitive function has declined exhibits a unique characteristic. Therefore, in the present disclosure, the voice information of the subject 5 is acquired, and it is determined whether the cognitive function has declined, based on whether the frequency characteristic exhibits the above-described unique characteristic. Therefore, the management server 1 can reduce processing of analyzing the content of the conversation uttered by the subject 5.

### MODIFIED EXAMPLE

Next, modified examples of the above example embodiment will be described. The following modified examples can be appropriately combined with the above example embodiment. In the above example embodiment, the case where the reaction information 7 is the voiceprint of the subject 5 has been described, but the reaction information 7 is not limited to the voiceprint. Variations of the reaction information 7 will be described below.

### First Modified Example

Hereinafter, a case where the reaction information 7 includes a facial video will be described as a first modified example. In the first modified example, the collection unit 28 of the dialogue device 2 includes a camera, and during a face-to-face conversation with the subject 5, the conversation implementation unit 51 controls the camera to capture a face of the subject 5, and transmits the reaction information 7 including the facial video obtained by the capturing and the acquisition date and time 7a of the facial video to the management server 1. In the management server 1, as in the above example embodiment, the cognitive function analysis unit 42 generates the analysis result 8 by using the reaction information 7 received from the dialogue device 2 and accumulated in the DB 15, and transmits the generated analysis result 8 to the dialogue device 2.

As the cognitive function check processing by the cognitive function analysis unit 42, for instance, a technique (Hereinafter, it is referred to as “technique G1”.) disclosed in International Application PCT/JP2023/041209 is used. That is, the cognitive function is evaluated by calculating time-series information regarding an eye opening degree of an eye in a section where it is determined that the subject 5 is in an awake state based on the facial video included in the reaction information 7, and calculating an eyelid variability feature based on the calculated time-series information. In addition, the cognitive function analysis unit 42 of the management server 1 may include technique G1 in addition to the cognitive function check processing of the present disclosure, and the accuracy of the cognitive function analysis processing can be improved based on the voice information and the facial video.

### Second Modified Example

In a second modified example, the reaction information 7 includes the facial video as in the first modified example, but instead of evaluating by the eye opening degree, the cognitive function is evaluated based on a facial expression of the subject 5. It is assumed that, as in the first modified example, the collection unit 28 of the dialogue device 2 includes the camera, and during the face-to-face conversation with the subject 5, the conversation implementation unit 51 transmits the reaction information 7 including the facial video obtained by controlling the camera and the acquisition date and time 7a of the facial video to the management server 1.

The cognitive function check processing by the cognitive function analysis unit 42 in the management server 1 executes processing of calculating a mouth corner variation speed from a mouth corner distance that is a distance from a mouth center to both ends of a mouth corner in a response state of the subject 5 and a variation amount of the mouth corner distance, and evaluating the cognitive function by using the calculated mouth corner variation speed, for instance, by using a technique (Hereinafter, it is referred to as “technique G2”.) disclosed in Japanese Patent Application No. 2024-049287. The management server 1 transmits the analysis result 8 obtained in this manner to the dialogue device 2. In addition, the cognitive function analysis unit 42 of the management server 1 may include technique G2 in addition to the cognitive function check processing of the present disclosure, and the accuracy of the cognitive function analysis processing can be improved based on the voice information and the facial video.

### Third Modified Example

In a third modified example, in addition to the evaluation of the cognitive function based on the facial video in the first modified example or the second modified example, the cognitive function may be evaluated based on walking data of the subject 5 measured using an insole provided with a sensor. In the third modified example, the reaction information 7 includes the facial video and the walking data. For instance, the walking data is acquired by causing the subject 5 to wear the insole provided with the sensor that can communicate with the dialogue device 2 by near field communication or the like and measures walking. A level of frailty is estimated using, for the acquired walking data, a technique (Hereinafter, it is referred to as “technique G3”.) disclosed in Japanese Patent Application No. 2024-078683.

Fureiru is a Japanese translation of Frailty (frailty), and is a concept proposed by The Japan Geriatrics Society in 2014. The level of frailty can be estimated based on the acquired walking data and physical information such as sex, date of birth, height, weight, and the like. The estimation result can be accumulated in the DB 15 as the check results 4b. The cognitive function analysis unit 42 generates the analysis result 8 by using the plurality of accumulated check results 4b as in the above example embodiment, and transmits the generated analysis result 8 to the dialogue device 2. In addition, the cognitive function analysis unit 42 of the management server 1 may include technique G3 in addition to the cognitive function check processing of the present disclosure, and the accuracy of the cognitive function analysis processing can be improved based on the voice information and the facial video.

Further, the cognitive function analysis unit 42 of the management server 1 may include at least two of techniques G1 to G3 in addition to the cognitive function check processing of the present disclosure. A more detailed analysis result 8 can be obtained. Therefore, according to the above example embodiment and the first to third modified examples, it is possible to acquire the reaction information 7 indicating a natural reaction excluding previous preparation of the subject 5 at an appropriate dialogue timing for obtaining the predetermined amount of the test data 4a in daily life. In addition, it is possible to provide healthcare for maintaining the cognitive function of the subject 5, by repeatedly having a dialogue on a daily basis. In addition, in the management server 1, the dialogue timing can be optimized by providing the dialogue model 4m, and the reaction information 7 can be efficiently collected and analyzed.

### Second Example Embodiment

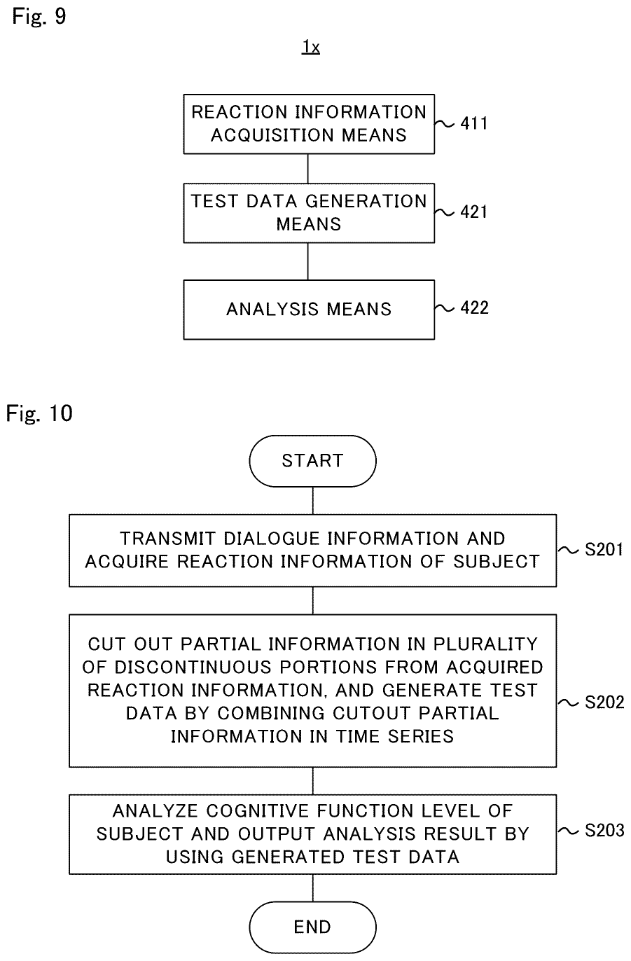

FIG. 9 is a block diagram illustrating a functional configuration of a management server according to a second example embodiment. A management server 1x includes a reaction information acquisition means 411, a test data generation means 421, and an analysis means 422. A hardware configuration of the management server 1x is similar to that of the first example embodiment. The reaction information acquisition means 411, the test data generation means 421, and the analysis means 422 are achieved by a processor 12 executing programs each associated to the above-described means.

FIG. 10 is a flowchart of processing by the management server of the second example embodiment. In the management server 1x, the reaction information acquisition means 411 transmits dialogue information 6 indicating a dialogue content to be uttered to a subject 5, and acquires reaction information 7 indicating a reaction of the subject 5 to the dialogue content (step S201). The dialogue information 6 is generated by a dialogue model 4m. The test data generation means 421 cuts out partial information in a plurality of discontinuous portions from the reaction information 7 acquired, and generates test data 4a by combining the cutout partial information in time series (step S202). The analysis means 422 analyzes a cognitive function level 4e of the subject 5 and outputs an analysis result by using the generated test data 4a (step S203).

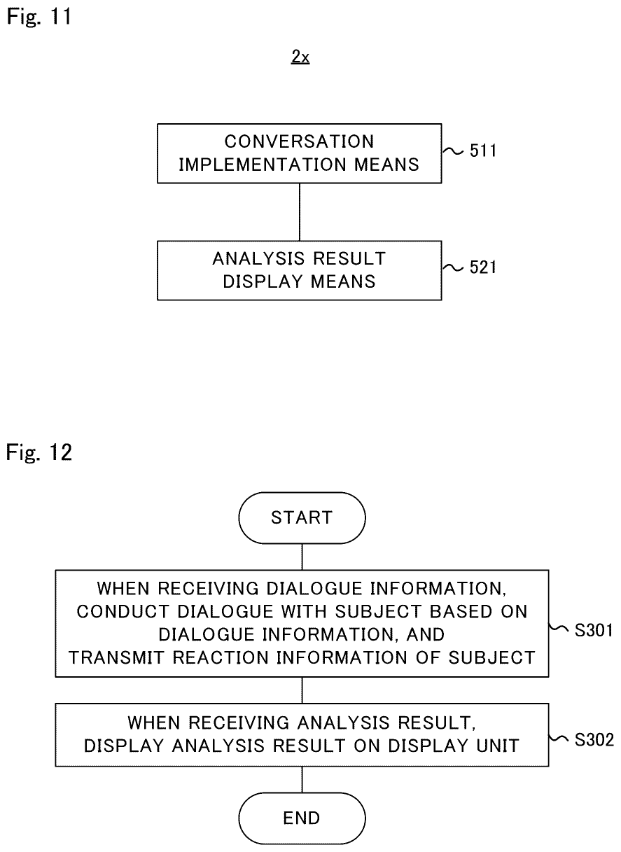

FIG. 11 is a block diagram illustrating a functional configuration of a dialogue device according to the second example embodiment. A dialogue device 2x includes a conversation implementation means 511 and an analysis result display means 521. A hardware configuration of the dialogue device 2x is similar to that of the first example embodiment. The conversation implementation means 511 and the analysis result display means 521 are achieved by the processor 22 executing programs each associated to the above-described means.

FIG. 12 is a flowchart of processing by the dialogue device of the second example embodiment. In the dialogue device 2x, in a case of receiving the dialogue information 6 from the management server 1x, the conversation implementation means 511 conducts a dialogue with the subject 5 based on the dialogue information 6, acquires the reaction information 7 of the subject 5, and transmits the reaction information 7 to the management server 1x (step S301). In a case of receiving the analysis result 8 from the management server 1x, the analysis result display means 521 displays the analysis result 8 on the display unit 26 (step S302).

As described above, according to the first example embodiment, the first to third modified examples, and the second example embodiment, a frequency of the number of conversations of the subject 5 can be increased in order to encourage the subject 5 to have a conversation. In addition, since the test data is generated in which the pieces of partial information in the plurality of discontinuous portions are combined in time series, the cognitive function level can be checked without depending on the content of the conversation by the subject 5. Therefore, the evaluation accuracy of the cognitive function can be improved. In addition, since the subject 5 who uses the dialogue device 2 or 2x can confirm the analysis result 8 of whether the subject 5 himself/herself has the tendency of the decline in cognitive function, it is possible to support the subject 5 in making a decision on whether to visit a medical institution. Furthermore, since the management servers 1 and 1x output the analysis result 8 indicating the daily cognitive function level of the subject 5, it is possible to support decision making in a case where a medical worker or the like performs a specialized diagnosis.

Some or all of the above example embodiments (including the modified examples, the same applies hereinafter) can also be described as the following Supplementary Notes, but are not limited to the following Supplementary Notes.

### (Supplementary Note 1)

An information processing device comprising:
a reaction information acquisition means configured to transmit dialogue information indicating a dialogue content to be uttered to a subject and acquire reaction information indicating a reaction of the subject to the dialogue content;
a test data generation means configured to cut out pieces of partial information in a plurality of discontinuous portions from the reaction information acquired and generate test data by combining in time series the pieces of partial information cut out; and
an analysis means configured to analyze a cognitive function level of the subject and output an analysis result by using the test data generated.

### (Supplementary Note 2)

The information processing device according to supplementary note 1, wherein
the reaction information acquisition means includes a dialogue model that estimates a dialogue timing of talking to the subject and the dialogue content by machine learning in such a way that the test data becomes data of a predetermined amount or more designated from the subject, and
the test data generated by the test data generation means becomes the data of the predetermined amount or more.

### (Supplementary Note 3)

The information processing device according to supplementary note 2, wherein the dialogue model estimates the dialogue timing in such a way that one or a plurality of dialogues are conducted for each of designated time periods.

### (Supplementary Note 4)

The information processing device according to supplementary note 3, wherein
the analysis means:
generates the test data for each of the designated time periods, executes cognitive function check processing of checking the cognitive function level of the subject based on the test data generated, and outputs a check result; and
executes tendency analysis processing of analyzing a tendency of a cognitive function of the subject based on a plurality of the check results obtained by the cognitive function check processing, and outputs the analysis result.

### (Supplementary Note 5)

The information processing device according to supplementary note 4, wherein the reaction information is voice information indicating a voiceprint in a case where the subject utters a voice with respect to the dialogue content uttered to the subject.

### (Supplementary Note 6)

The information processing device according to supplementary note 4, wherein
the reaction information includes a facial video, and
the analysis means executes processing of evaluating the cognitive function by calculating time-series information regarding an eye opening degree of an eye in a section where it is determined that the subject is in an awake state based on the facial video, and calculating an eyelid variability feature based on the time-series information calculated, and outputs an analysis result regarding the processing.

### (Supplementary Note 7)

The information processing device according to supplementary note 4, wherein
the reaction information includes a facial video, and
the analysis means executes processing of calculating a mouth corner variation speed from a mouth corner distance that is a distance from a mouth center to both ends of a mouth corner in a response state of the subject and a variation amount of the mouth corner distance, and evaluating the cognitive function by using the mouth corner variation speed calculated, and outputs an analysis result regarding the processing.

### (Supplementary Note 8)

The information processing device according to supplementary note 4,wherein
the reaction information includes a facial video and walking data, and
the analysis means executes processing of estimating the cognitive function based on the facial video and the walking data, and outputs an analysis result regarding the processing.

### (Supplementary Note 9)

A terminal device connectable to the information processing device according to supplementary note 1 via a network, the terminal device comprising:
a conversation implementation means configured to, in a case of receiving the dialogue information from the information processing device, conduct a dialogue with the subject based on the dialogue information, acquire the reaction information of the subject, and transmit the reaction information to the information processing device; and
an analysis result display means configured to, in a case of receiving the analysis result from the information processing device, display the analysis result on a display unit.

### (Supplementary Note 10)

An information processing method performed by a computer, the method comprising:
transmitting dialogue information indicating a dialogue content to be uttered to a subject and acquiring reaction information indicating a reaction of the subject to the dialogue content;
cutting out pieces of partial information in a plurality of discontinuous portions from the reaction information acquired and generating test data by combining in time series the pieces of partial information cut out; and
analyzing a cognitive function level of the subject and output an analysis result by using the test data generated.

### (Supplementary Note 11)

A program causing a computer to execute processing of:
transmitting dialogue information indicating a dialogue content to be uttered to a subject and acquiring reaction information indicating a reaction of the subject to the dialogue content;
cutting out pieces of partial information in a plurality of discontinuous portions from the reaction information acquired and generating test data by combining in time series the pieces of partial information cut out; and
analyzing a cognitive function level of the subject and output an analysis result by using the test data generated.

Some or all of the configurations described in Supplementary Notes 2 to 8 dependent on the above-described Supplementary Note 1 can also be dependent on Supplementary Notes 10 and 11 by the same dependency relationship as in Supplementary Notes 2 to 9. Furthermore, some or all of the configurations described as Supplementary Notes can be similarly dependent on not only Supplementary Notes 1, 10, and 11, but also various pieces of hardware and software, and various recording means or systems for recording software without departing from the above-described example embodiments.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. That is, it is a matter of course that the present disclosure includes various modifications and corrections that can be made by those of ordinary skill in the art in accordance with the entire disclosure including the claims and the technical idea.

### DESCRIPTION OF SYMBOLS

1 Management server
2 Dialogue device
4m Dialogue model
5 Subject
41 Conversation management unit
42 Cognitive function analysis unit
51 Conversation implementation unit
52 Analysis result display unit

1. An information processing device comprising:
at least one first memory configured to store first instructions; and
at least one first processor configured to execute the first instructions to:
transmit dialogue information indicating a dialogue content to be uttered to a subject and acquire reaction information indicating a reaction of the subject to the dialogue content;
cut out pieces of partial information in a plurality of discontinuous portions from the reaction information acquired and generate test data by combining in time series the pieces of partial information cut out; and
analyze a cognitive function level of the subject and output an analysis result by using the test data generated.

2. The information processing device according to claim 1, wherein
to transmit the dialogue information, the first processor uses a dialogue model that estimates a dialogue timing of talking to the subject and the dialogue content by machine learning in such a way that the test data becomes data of a predetermined amount or more designated from the subject, and
the test data generated by the first processor becomes the data of the predetermined amount or more.

3. The information processing device according to claim 2, wherein the dialogue model estimates the dialogue timing in such a way that one or a plurality of dialogues are conducted for each of designated time periods.

4. The information processing device according to claim 3, wherein
to analyze the cognitive function level of the subject, the first processor is further configured to:
generate the test data for each of the designated time periods, executes cognitive function check processing of checking the cognitive function level of the subject based on the test data generated, and output a check result; and
execute tendency analysis processing of analyzing a tendency of a cognitive function of the subject based on a plurality of the check results obtained by the cognitive function check processing, and output the analysis result.

5. The information processing device according to claim 4, wherein the reaction information is voice information indicating a voiceprint in a case where the subject utters a voice with respect to the dialogue content uttered to the subject.

6. The information processing device according to claim 4, wherein
the reaction information includes a facial video, and
the first processor executes processing of evaluating the cognitive function by calculating time-series information regarding an eye opening degree of an eye in a section where it is determined that the subject is in an awake state based on the facial video, and calculating an eyelid variability feature based on the time-series information calculated, and outputs an analysis result regarding the processing.

7. The information processing device according to claim 4, wherein
the reaction information includes a facial video, and
the first processor executes processing of calculating a mouth corner variation speed from a mouth corner distance that is a distance from a mouth center to both ends of a mouth corner in a response state of the subject and a variation amount of the mouth corner distance, and evaluating the cognitive function by using the mouth corner variation speed calculated, and outputs an analysis result regarding the processing.

8. A terminal device connectable to the information processing device according to claim 1 via a network, the terminal device comprising:
at least one second memory configured to store second instructions; and
at least one second processor configured to execute the second instructions to:
conduct a dialogue with the subject based on the dialogue information, acquire the reaction information of the subject, and transmit the reaction information to the information processing device in a case of receiving the dialogue information from the information processing device; and
display the analysis result on a display unit in a case of receiving the analysis result from the information processing device.

9. An information processing method performed by a computer, the method comprising:
transmitting dialogue information indicating a dialogue content to be uttered to a subject and acquiring reaction information indicating a reaction of the subject to the dialogue content;
cutting out pieces of partial information in a plurality of discontinuous portions from the reaction information acquired and generating test data by combining in time series the pieces of partial information cut out; and
analyzing a cognitive function level of the subject and output an analysis result by using the test data generated.

10. A non-transitory computer-readable recording medium storing a program causing a computer to execute processing of:
transmitting dialogue information indicating a dialogue content to be uttered to a subject and acquiring reaction information indicating a reaction of the subject to the dialogue content;
cutting out pieces of partial information in a plurality of discontinuous portions from the reaction information acquired and generating test data by combining in time series the pieces of partial information cut out; and
analyzing a cognitive function level of the subject and output an analysis result by using the test data generated.
